(19) United States
(12) Patent Application Publication
(10) Pub. No.: US 2021/0102152 A1
(43) Pub. Date: Apr. 8, 2021

(54) BEAD BIOFILM REACTOR

(71) Applicant: University of Utah Research Foundation, Salt Lake City, UT (US)

(72) Inventors: Nicholas N. Ashton, Salt Lake City, UT (US); Dustin L. Williams, Salt Lake City, UT (US)

(21) Appl. No.: 17/011,791

(22) Filed: Sep. 3, 2020

Related U.S. Application Data
(60) Provisional application No. 62/895,451, filed on Sep. 3, 2019.

(51) Int. Cl. C12M 1/12; C12M 1/00; C12M 3/00; C12M 1/34

(52) U.S. Cl. CPC C12M 25/14; C12M 23/06; C12M 41/46; C12M 23/44; C12M 25/16; C12M 23/28

(57) ABSTRACT

A biofilm reactor substrate support system can comprise a base holder shaped to retain a reactor vessel and comprising a magnetic alignment feature. The biofilm reactor substrate can also comprise a set of reactor racks including a complementary magnetic alignment feature which can be magnetically coupleable to the magnetic alignment feature so as to anchor the base holder and the set of reactor racks in a fixed position relative to one another. Each reactor rack can be coupleable to at least one removable biofilm support microstructure.

### RELATED APPLICATION

This application claims the benefit of U.S. Provisional Patent Application Ser. No. 62/895,451, filed on Sep. 3, 2019, the entire contents of which are incorporated herein by reference.

### FIELD OF THE INVENTION

The present invention relates to testing and screening anti-biofilm compounds and bacterio-physiology research. Therefore, the present invention relates generally to the fields of biology, cell physiology, immunology, and material science.

### BACKGROUND

Biofilms can form on surfaces and can be found in natural, industrial, and hospital settings. The microbial cells growing in a biofilm are physiologically distinct from planktonic cells of the same organism, which, by contrast, are single-cells that may float or swim in a liquid medium. The testing and screening of compounds for use against biofilms can be difficult because of inconsistencies in environmental conditions among the biofilms tested and treated.

For testing and screening anti-infection devices and bactericidal, antibiotic, and anti-infective compounds, researchers and the biotech industry rely on the use of actively-dividing planktonic bacterial phenotypes in assays such as the minimum inhibitory concentration (MIC), the E-test, Kirby-Bauer, the minimum bactericidal concentration (MBC), and MicroScan. Yet quiescent, non-motile, bacterial phenotypes are implicated in some of the more harmful clinical infections. The pathogens causing these infections excrete sticky exopolysaccharides to form cohesive communal aggregates and adhesive attachments to foreign surfaces like devitalized tissues and implanted biomaterials, which can negatively impact phagocytic clearance by host immune cells. The quiescent phenotypic variants in these bacterial communities are tolerant of antibiotic concentrations many orders of magnitude greater than the concentrations of antibiotics that can kill planktonic phenotypes used in conventional assays. Consequently, the antibiotic concentrations that are effective in killing planktonic phenotypes can exceed toxic thresholds that bound safe systemic antibiotic concentrations. The use of biofilms in assays for screening candidate compounds can identify anti-infection compounds and combination therapies that may not be predicted from the minimum inhibitory concentration (MIC) assay. The use of existing biofilm reactors for susceptibility assays and related tests is cumbersome, uses excessive materials, uses excessive culture medium, uses excessive amounts of expensive antibiotic compounds, and involves a time-intensive approach. These reactor systems were not designed to test for screening candidate compounds to identify anti-infective compounds.

For example, some biofilm reactors include: (a) The Center for Disease Control (CDC) biofilm reactor, and (b) the drip-flow biofilm. There is also a biofilm assay (e.g., the MBEC Assay®) that includes a 96 well plate. These systems use copious amounts of expensive culture medium and have considerable dead-volumes (i.e. high volumes of broth). Once the biofilms are grown, treatments are typically performed on samples which have been moved, by necessity, to secondary treatment vessels (e.g., a test tube or laboratory beaker). In these treatment vessels there is a drastic change in environmental variables such as: (i) flow, (ii) broth turnover rate, and (iii) sheer forces. Effects from these unintended factors complicate the analysis of the effects of the treatments.

When treatments are applied in the reactors, the experiments can use an additional amount of culture medium and antibiotics to be loaded into the culture medium that are perfused through the reactor at target therapeutic concentrations. The amount of antibiotics can be too expensive. For example, a CDC biofilm reactor can use about 13 L of broth for a 48-hour growth cycle. If antibiotic compounds were to be tested using this setup, and an assumed amount of 5 mg/ml were loaded in the system, then a single test would use about 65 grams of material. For inexpensive antibiotics this would be several hundred dollars, but for experimental compounds this amount of antibiotics would exceed tens of thousands of dollars for just one test.

The MBEC assay may use fewer reagents and material but presents other issues. First, the MBEC assay does not produce the metabolically quiescent core of established dense biofilms as in a CDC biofilm reactor system. Second, the biofilms in the MBEC assay can vary across the wells of the assay which introduces data that can be unreliable and difficult to reproduce. Third, the setup of the MBEC assay is disposable, non-reusable, and expensive for screening many compounds. Consequently, the aforementioned biofilm reactors and assays ignore the disease etiology of biofilms and do not provide adequate results.

### SUMMARY

A biofilm reactor and substrate system can facilitate quicker and low-cost screening of candidate compounds or combination therapies using robust biofilms with high surface-area density. More generally, the biofilm reactor disclosed herein can facilitate bacterio-physiology studies.

In one embodiment, a biofilm reactor substrate support system can comprise a base holder shaped to retain a reactor vessel. The base holder can comprise a magnetic alignment feature. The biofilm reactor substrate support system can further comprise a set of reactor racks including a complementary magnetic alignment feature which can be magnetically coupleable to the magnetic alignment feature so as to anchor the base holder and the set of reactor racks in a fixed position relative to one another. In one aspect, each reactor rack can be coupleable to at least one removable biofilm support microstructure.

In another embodiment, a method for screening anti-biofilm compounds can comprise anchoring a set of reactor racks in a fixed position to a base holder using a magnetic alignment feature and a complementary magnetic alignment feature. In one example, the method can comprise growing biofilm on a removable biofilm support structure coupled to a reactor rack of the set of reactor racks. In another example, the method can comprise separating the removable biofilm support structure and the reactor rack from the set of reactor racks. In one aspect, the method can comprise treating the biofilm on the removable support structure with an anti-biofilm compound.

There has thus been outlined, rather broadly, the more important features of the invention so that the detailed description thereof that follows may be better understood, and so that the present contribution to the art may be better appreciated. Other features of the present invention will become clearer from the following detailed description of the invention, taken with the accompanying drawings and claims, or may be learned by the practice of the invention.

### BRIEF DESCRIPTION OF THE DRAWINGS

Features and advantages of the disclosure will be apparent from the detailed description which follows, taken in conjunction with the accompanying drawings, which together illustrate, by way of example, features of the disclosure.

FIG. 1 is a top-down view showing how the magnetic base holder can secure a set of racks at the outer edge of the reactor dish in accordance with an example.

FIG. 2a is a representative reactor rack with five removable biofilm support microstructures mounted on each of their holder pins in accordance with an example.

FIG. 2b is a representative reactor rack with the five vertical holder pins and the unmounted removable biofilm support microstructures for growing biofilms in accordance with an example.

FIG. 2c illustrates various interface mechanisms of coupling the removable biofilm support microstructures to the representative reactor rack in accordance with an example.

FIG. 2d illustrates various representative shapes of removable biofilm support microstructures for growing biofilms in accordance with an example.

FIG. 3 illustrates a reactor dish with the six reactor racks, thirty vertical holder pins, and thirty removable biofilm support microstructures mounted on each of the associated vertical holder pins in accordance with an example.

FIG. 4 is a perspective view of the reactor dish with the six racks, thirty pins, and corresponding mounted removable biofilm support microstructures in accordance with an example.

FIG. 5 illustrates a base holder for holding the reactor dish in which the black dots illustrate embedded neodymium magnets in accordance with an example.

FIG. 6 illustrates a reactor dish and reactor components mounted into the magnetic base in which the reactor racks are each centered above a corresponding neodymium magnet in accordance with an example.

FIG. 7 illustrates a magnetic base holder securing a single rack at the outer edge of the reactor dish in accordance with an example.

FIG. 8 illustrates a top-down view showing how the magnetic base holder can secure a set of five racks at the outer edge of the reactor dish in accordance with an example.

FIG. 9 illustrates the biofilms on the substrate beads after a time period of growth in accordance with an example.

FIG. 10 illustrates the removable biofilm support microstructures and associated biofilms in a test tube for biofilm quantification in accordance with an example.

FIG. 11a illustrates the base holder with one representative rack and threaded connectors in accordance with an example.

FIG. 11b illustrates a top holder with three threaded retaining rings in accordance with an example.

FIG. 11c illustrates the base holder coupled to the top holder with three wing nuts in accordance with an example.

FIG. 12 is a flowchart depicting a method for screening antibiofilm compounds in accordance with an example.

FIG. 13 depicts a graph comparison of equivalent 48 h biofilms grown with S. aureus ATCC 6538 with the CDC biofilm reactor and the biofilm reactor described herein in accordance with an example.

These drawings are provided to illustrate various aspects of the invention and are not intended to be limiting of the scope in terms of dimensions, materials, configurations, arrangements or proportions unless otherwise limited by the claims.

### DETAILED DESCRIPTION

While these exemplary embodiments are described in sufficient detail to enable those skilled in the art to practice the invention, it should be understood that other embodiments may be realized and that various changes to the invention may be made without departing from the spirit and scope of the present invention. Thus, the following more detailed description of the embodiments of the present invention is not intended to limit the scope of the invention, as claimed, but is presented for purposes of illustration only and not limitation to describe the features and characteristics of the present invention, to set forth the best mode of operation of the invention, and to sufficiently enable one skilled in the art to practice the invention. Accordingly, the scope of the present invention is to be defined solely by the appended claims.

### Definitions

In describing and claiming the present invention, the following terminology will be used.

The singular forms “a,” “an,” and “the” include plural referents unless the context clearly dictates otherwise. Thus, for example, reference to “a particle” includes reference to one or more of such materials and reference to “subjecting” refers to one or more such steps.

As used herein, the term “about” is used to provide flexibility and imprecision associated with a given term, metric or value. The degree of flexibility for a particular variable can be readily determined by one skilled in the art. However, unless otherwise enunciated, the term “about” generally connotes flexibility of less than 2%, and most often less than 1%, and in some cases less than 0.01%.

As used herein with respect to an identified property or circumstance, “substantially” refers to a degree of deviation that is sufficiently small so as to not measurably detract from the identified property or circumstance. The exact degree of deviation allowable may in some cases depend on the specific context.

As used herein, “adjacent” refers to the proximity of two structures or elements. Particularly, elements that are identified as being “adjacent” may be either abutting or connected. Such elements may also be near or close to each other without necessarily contacting each other. The exact degree of proximity may in some cases depend on the specific context.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

As used herein, the term “at least one of” is intended to be synonymous with “one or more of.” For example, “at least one of A, B and C” explicitly includes only A, only B, only C, and combinations of each.

Concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a numerical range of about 1 to about 4.5 should be interpreted to include not only the explicitly recited limits of 1 to about 4.5, but also to include individual numerals such as 2, 3, 4, and sub-ranges such as 1 to 3, 2 to 4, etc. The same principle applies to ranges reciting only one numerical value, such as “less than about 4.5,” which should be interpreted to include all of the above-recited values and ranges. Further, such an interpretation should apply regardless of the breadth of the range or the characteristic being described.

Any steps recited in any method or process claims may be executed in any order and are not limited to the order presented in the claims. Means-plus-function or step-plus-function limitations will only be employed where for a specific claim limitation all of the following conditions are present in that limitation: a) “means for” or “step for” is expressly recited; and b) a corresponding function is expressly recited. The structure, material or acts that support the means-plus function are expressly recited in the description herein. Accordingly, the scope of the invention should be determined solely by the appended claims and their legal equivalents, rather than by the descriptions and examples given herein.

Biofilm Reactor

An initial overview of invention embodiments is provided below and specific embodiments are then described in further detail. This initial summary is intended to aid readers in understanding the technological concepts more quickly, but is not intended to identify key or essential features thereof, nor is it intended to limit the scope of the claimed subject matter.

The biofilm reactor substrate support system allows less broth, fewer antibiotic reagents, and fewer culture tubes for acquiring antibiotic susceptibility testing on established biofilms. The biofilm reactor may not use the expensive peristaltic pumps that are used in the drip-flow and CDC biofilm reactors. Further, the biofilm reactor can fit into an anaerobic chamber jar which can make the growth of anaerobic bacteria cheaper and less equipment intensive than other biofilm reactor types.

In one embodiment, the biofilm reactor substrate support system can comprise a base holder shaped to retain a reactor vessel. The base holder can comprise a magnetic alignment feature. The biofilm reactor substrate support system can further comprise a set of reactor racks including a complementary magnetic alignment feature which can be magnetically coupleable to the magnetic alignment feature so as to anchor the base holder and the set of reactor racks in a fixed position relative to one another. In one aspect, each reactor rack can be coupleable to at least one removable biofilm support microstructure.

In another embodiment, a method for screening anti-biofilm compounds can comprise anchoring a set of reactor racks in a fixed position to a base holder using a magnetic alignment feature and a complementary magnetic alignment feature. In one example, the method can comprise growing biofilm on a removable biofilm support structure coupled to a reactor rack of the set of reactor racks. In another example, the method can comprise separating the removable biofilm support structure and the reactor rack from the set of reactor racks. In one aspect, the method can comprise treating the biofilm on the removable support structure with an anti-biofilm compound.

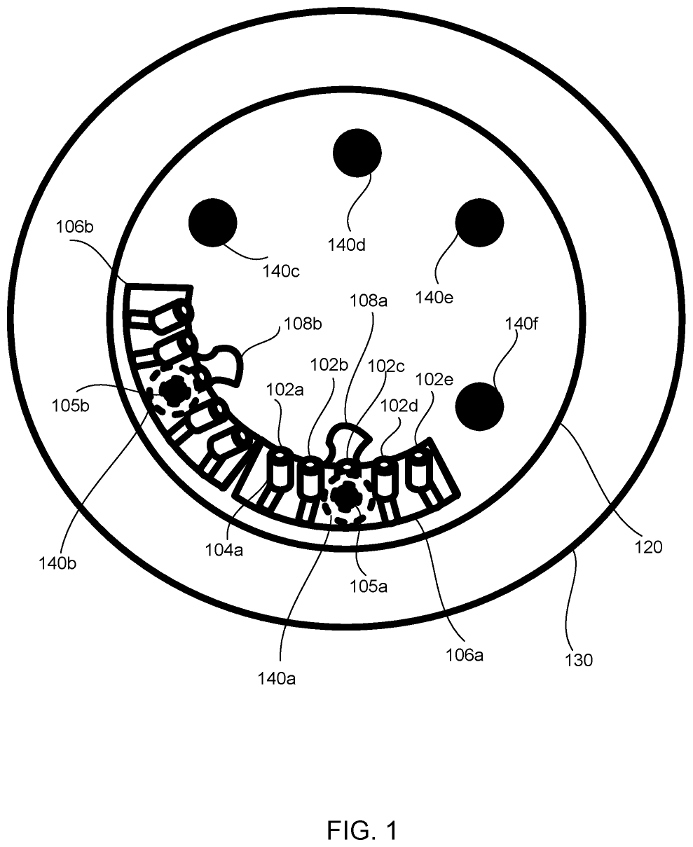

In one example, as illustrated with reference to FIG. 1, a biofilm reactor substrate support system 100 can comprise a base holder 130 shaped to retain a reactor vessel 120. In one aspect, the base holder 130 can include a magnetic alignment feature (e.g., 140a, 140b, 140c 140d, 140e, and 140f). In another aspect, the biofilm reactor substrate support system 100 can comprise a set of reactor racks (e.g., 106a, 106b). The set of reactor racks 106a-b can include a complementary magnetic alignment feature (e.g., 105a and 105b) which can be magnetically coupled to the magnetic alignment feature 140a, 140b, 140c 140d, 140e, and 140f of the base holder 130 so as to anchor the base holder 130 and the set of reactor racks 106a-b in a fixed position relative to one another. In another alternative, the set of reactor racks can be fixed relative to the base holder using suction cups as the alignment feature. For example, suction cups can be coupled to a bottom surface of each of the reactor racks to secure the racks relative to a reactor vessel (e.g. glassware). The base holder can then be shaped to receive the reactor vessel in a fixed position so that the base holder and reactor racks are fixed in position relative to one another.

In another aspect, each reactor rack (e.g., 106a, 106b) in the set of reactor racks 106a-106b can be coupled to at least one removable biofilm support microstructure (e.g., 102a, 102b, 102c, 102d, or 102e).

In one example, the set of reactor racks 106a-106b can include between about 2 and about 10 reactor racks. Each individual reactor rack (e.g., 106a, 106b) can be coupled to the base holder 130 with the reactor vessel 120 positioned intermediate.

In another example, each individual reactor rack (e.g., 106a, 106b) can be connectable with at least one adjacent reactor rack (e.g., 106a and 106b can be adjacent) in the set of reactor racks 106a-106b. In one aspect, the set of reactor racks 106a-106b can be configured to be uniformly distributed inside the reactor vessel 120 when each reactor rack (e.g., 106a, 106b) is connected with the adjacent reactor rack (e.g., 106a, 106b). In another example, each reactor rack (e.g., 106a, 106b) can have an arcuate shape that matches a bottom perimeter of the reactor vessel 120. In another example, each reactor rack (e.g., 106a, 106b) can comprise a material including at least one of: stainless steel.

In another example, the magnetic alignment feature (e.g., 140a, 140b, 140c 140d, 140e, and 140f) can be a set of permanent magnets and the complementary magnetic alignment feature (e.g., 105a and 105b) can be at least one of ferromagnetic material and ferrimagnetic material. The set of reactor racks 106a-106b can be either formed of or incorporate the complementary magnetic alignment feature (e.g., 105a and 105b).

In another example, the complementary magnetic alignment feature (e.g., 105a and 105b) can be a set of permanent magnets and the magnetic alignment feature (e.g., 140a, 140b, 140c 140d, 140e, and 140f) can be at least one of ferromagnetic material and ferrimagnetic material. In one aspect, the base holder 130 can be either formed of or incorporate the complementary magnetic alignment feature (e.g., 105a and 105b).

In another example, the biofilm reactor substrate support system 100 can further comprise at least one removable biofilm support microstructure (e.g., 102a, 102b, 102c, 102d, or 102e). In one aspect, the removable biofilm support microstructure 102a, 102b, 102c, 102d, or 102e) can have a width of less than about 10 mm. In one aspect, the removable biofilm support microstructure 102a, 102b, 102c, 102d, or 102e) can be shaped as at least one of: a bead, a cylinder, a sphere, a torus, an ellipsoid, or a helix. In one aspect, the removable biofilm support microstructure 102a, 102b, 102c, 102d, or 102e can be radially symmetric. In another aspect, the removable biofilm support microstructure 102a, 102b, 102c, 102d, or 102e can comprise one or more of glass, polymer, ceramic, or metal.

In another example, the removable biofilm support microstructure 102a, 102b, 102c, 102d, or 102e can comprise between about 1 and about 100 removable biofilm support microstructures. In another aspect, the removable biofilm support microstructures 102a, 102b, 102c, 102d, or 102e can be configured to be uniformly distributed along a peripheral perimeter of the reactor vessel 120 when coupled to the set of reactor racks 106a-106b.

In another example, the biofilm reactor substrate support system 100 can comprise a mount configured to receive the removable biofilm support microstructure 102a, 102b, 102c, 102d, or 102e. In one aspect, the mount can be a vertical pin and the removable biofilm support microstructure 102a, 102b, 102c, 102d, or 102e can include a receiver hole sized to be mounted on the vertical pin.

In another example, each reactor rack 106a, 106b can be coupled to at least one removable biofilm support microstructure 102a, 102b, 102c, 102d, or 102e using at least one or more of magnetic coupling, slotted coupling, snaps, interference fitting, adhesion, and threading.

In another example, the removable biofilm support microstructure 102a, 102b, 102c, 102d, or 102e can provide a biofilm growth surface area of 1 to 1000 mm² and a reactor volume of 10 to 2000 ml.

In another example, the biofilm reactor substrate support system 100 can comprise a reactor vessel 120 configured to contain the set of reactor racks 106a-106b. In another example, the set of reactor racks 106a-106b can each include a holding tab (e.g., 108a and 108b) configured to facilitate movement of the ferromagnetic reactor rack (e.g., 106a and 106b).

In another example, the biofilm reactor substrate support system 100 can include other components including but not limited to one or more of: a stir blade, silicone tubing, a carboy, a carboy lid, a magnetic stir plate, a pipette, a micropipette, a probe, an orbital shaker, a vortex shaker, wooden applicator sticks, a hemostat, an inoculating loop, culture tubes, a sterilizer or autoclave, a colony counter, a clamp, a petri dish, a glass flow break, a plexiglass board, the like, and combinations thereof. In another example, the biofilm reactor substrate support system 100 can be used with various media and reagants including but not limited to one or more of: culture media, buffer, plating media, ethanol, the like, and combinations thereof.

In another example, the biofilms can be formed from one or more of: gram-positive bacteria (e.g. Bacillus spp, Listeria monocytogenes, Staphylococcus spp, Lactobacillus plantarum, and Lactococcus lactis), gram-negative bacteria (e.g. Escherichia coli, or Pseudomonas aeruginosa), cyanobacteria, archaea, fungi, and microalgae. Some representative biofilm organisms include: Pseudomonas aeruginosa, and Streptococcus pneumoniae.

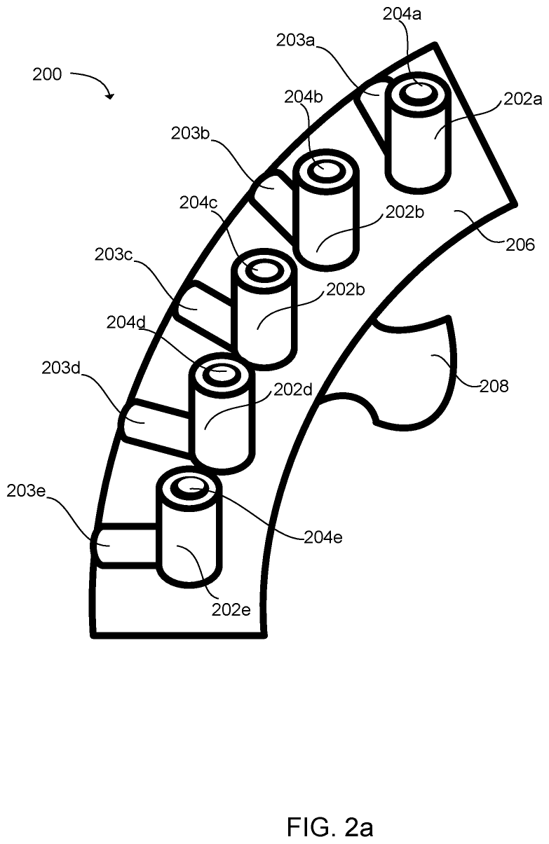

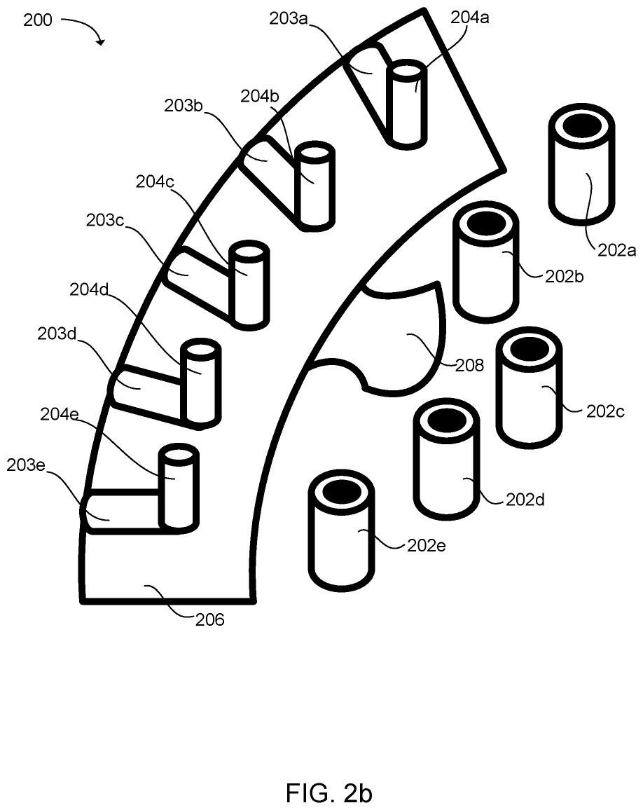

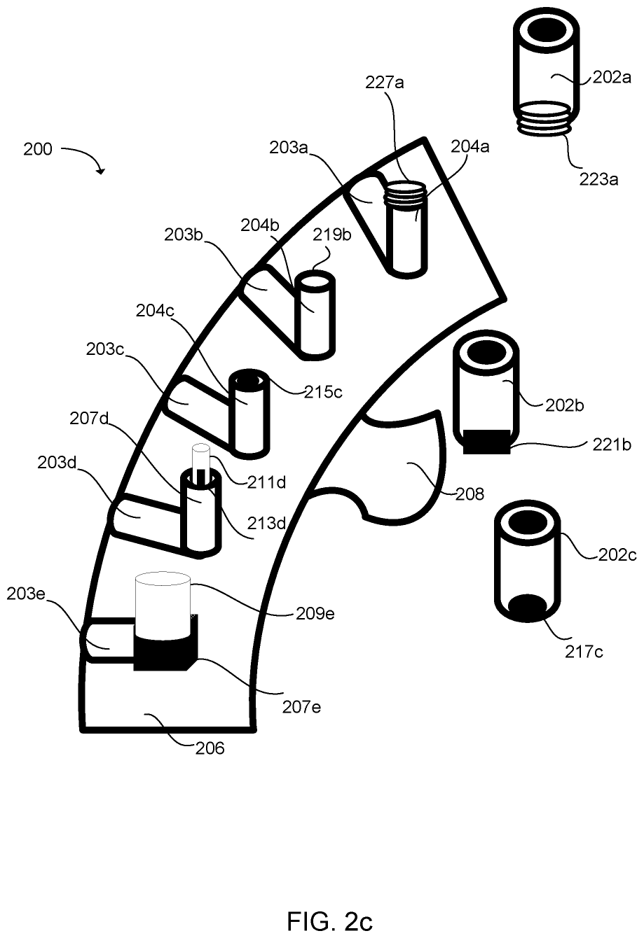

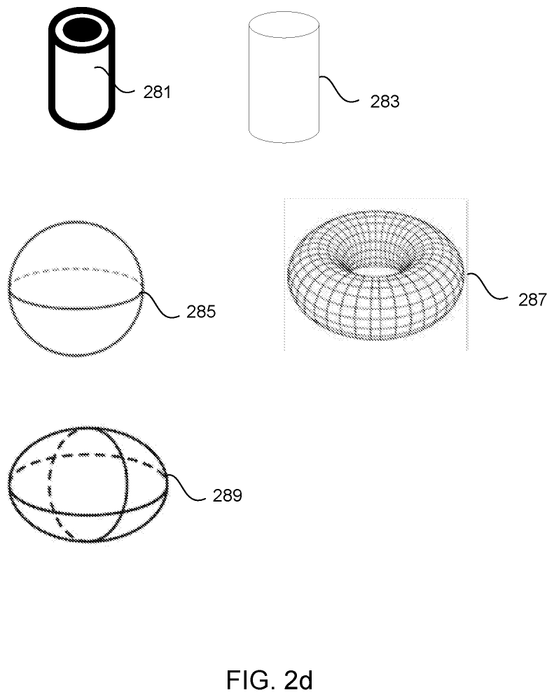

In another example, as illustrated in FIG. 2a, the biofilm reactor substrate support system 200 can further comprise at least one removable biofilm support microstructure (e.g., 202a, 202b, 202c, 202d, or 202e). In one aspect, the removable biofilm support microstructure 202a, 202b, 202c, 202d, or 202e can have a width of less than about 10 mm, or about 5 mm, or about 1 mm, or about 100 μm. In one aspect, the removable biofilm support microstructure 202a, 202b, 202c, 202d, or 202e can be shaped as at least one of: a bead, a cylinder, a sphere, a torus, an ellipsoid, and a helix. In another embodiment, the removable biofilm support microstructure 202a, 202b, 202c, 202d, or 202e can be shaped as at least one of: a cuboid, a cone, a tetrahedron, a triangular prism, a cube, a square pyramid, a pentagon, a hexagon, an octagon, and the like.

In another aspect, the removable biofilm support microstructure 202a, 202b, 202c, 202d, or 202e can comprise one or more of glass, polymer, ceramic, or metal. In another aspect, the removable biofilm support microstructure 202a, 202b, 202c, 202d, or 202e can comprise glass including but not limited to: borosilicate glass, quartz glass, actinic glass, fritted glass, coated glass, or siliconized glass.

In another aspect, the removable biofilm support microstructure 202a, 202b, 202c, 202d, or 202e can comprise a polymer including but not limited to: synthetic polymers or naturally-occurring polymers. Synthetic polymers can include but are not limited to: α-hydroxyacid, polyanhydrides, and the like. Naturally-occurring polymers can include but are not limited to: Polyvinylchloride (PVC), Polyethylene (PE), Polypropylene (PP), Polymethylmetacrylate (PMMA), Polystyrene (PS), Polytetrafluoroethylene (PTFE), Polyurethane (PU), Polyamide (nylon), Polyethylenterephthalate (PET), Polyethersulfone (PES), Polyetherimide (PEI), Polyetheretherketone (PEEK), the like, and combinations thereof.

In another aspect, the removable biofilm support microstructure 202a, 202b, 202c, 202d, or 202e can comprise a ceramic including but not limited to: Alumina (Al₂O₃), Zirconia (ZrO₂), Bioglass (Na₂OCaOP₂O₃—SiO), Hydroxyapatite, Tricalcium Phosphate, Polymeric, the like, and combinations thereof.

In another aspect, the removable biofilm support microstructure 202a, 202b, 202c, 202d, or 202e can comprise a metal including but not limited to: stainless steel, cobalt-chrome alloy, titanium, and nickel-titanium alloy (nitinol), gold, platinum, silver, iridium, tantalum, tungsten, the like, and combinations thereof.

In another example, the number of removable biofilm support microstructure in a reactor rack can be between 1 and about 100 removable biofilm support microstructures. In another example, the removable biofilm support microstructure can comprise between about 10 and about 50 removable biofilm support microstructures. In another example, the removable biofilm support microstructure can comprise between about 20 and about 40 removable biofilm support microstructures.

In one aspect, the removable biofilm support microstructure 202a, 202b, 202c, 202d, or 202e can be radially symmetric. A removable biofilm support microstructure that is radially symmetric can provide common environmental conditions (e.g., flow, broth turnover rate, and sheer forces) which can reduce the variance among removable biofilm support microstructures.

In another example, as illustrated in FIG. 2b, the biofilm reactor substrate support system 100 can comprise a mount (e.g., 204a, 204b, 204c, 204d, 204e, or 204f) configured to receive the removable biofilm support microstructure 202a, 202b, 202c, 202d, or 202e. In one aspect, the mount 204a, 204b, 204c, 204d, or 204e can be a vertical pin and the removable biofilm support microstructures 202a, 202b, 202c, 202d, or 202e can include a receiver hole sized to be mounted on the vertical pin.

In another example, as illustrated in FIG. 2c, a reactor rack 206 can be coupled to at least one removable biofilm support microstructure 209e, 211d, 202c, 202b, or 202a using at least one or more of magnetic coupling 207e, slotted coupling 207d and 213d, snaps 215c and 217c, interference fitting, adhesion 219b and 221b, and threading 227a and 223a. In order to maintain the consistency of environmental conditions, the type of coupling between a reactor rack and the removable biofilm support microstructure can be identical (e.g., each reactor rack can be coupled using one type of coupling without other types).

In another example, as illustrated in FIG. 2d, the removable biofilm support microstructure can have various shapes including: a bead 281, a cylinder 283, a sphere 285, a torus 287, or an ellipsoid 289. In another embodiment, the removable biofilm support microstructure 202a, 202b, 202c, 202d, or 202e can be shaped as at least one of: a helix, a cuboid, a cone, a tetrahedron, a triangular prism, a cube, a square pyramid, a pentagon, a hexagon, an octagon, and the like. Symmetrical shapes can present uniform environmental conditions (e.g., flow, broth turnover rate, and sheer forces) that can reduce the variance of the results.

In another example, the removable biofilm support microstructure 202a, 202b, 202c, 202d, or 202e can provide a biofilm surface area of 1 to 1000 mm² and a reactor volume of 10 to 2000 ml.

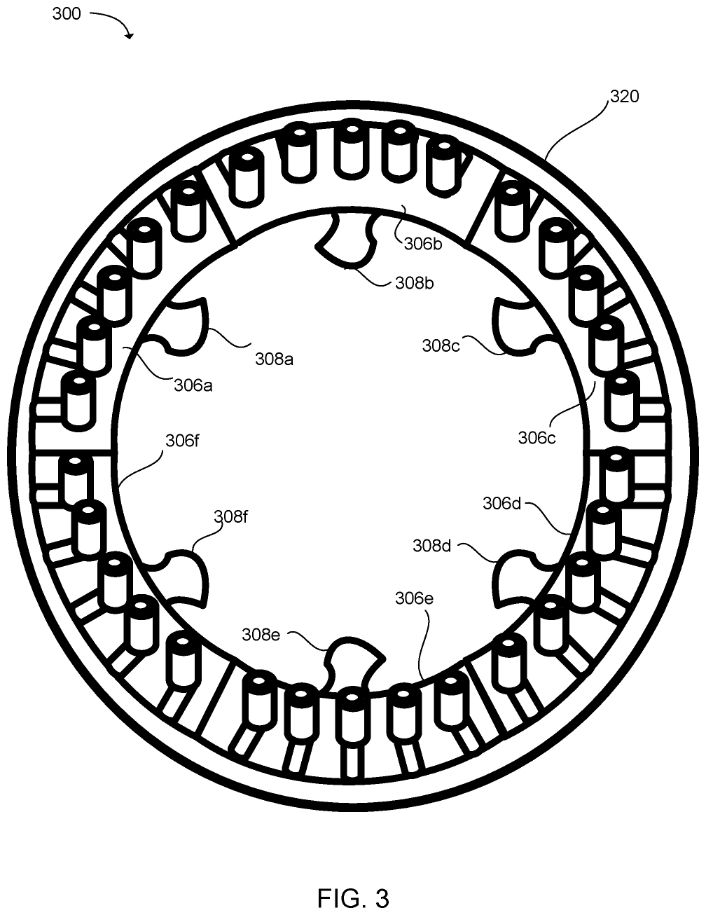

In another embodiment, as illustrated in FIG. 3 a reactor dish can include 6 reactor racks 306a-f, 30 vertical holder pins, and 30 removable biofilm support microstructures mounted on each of the associated vertical holder pins. Each of the 6 reactor racks can include a holding tab (e.g., 308a-f) configured to facilitate movement of the ferromagnetic reactor rack without substantially modifying the environmental conditions associated with each reactor rack.

In another example, the biofilm reactor substrate support system 300 can comprise a reactor vessel 320 configured to contain the set of reactor racks 306a-306f. In another aspect, the removable biofilm support microstructures can be configured to be uniformly distributed along a peripheral perimeter of the reactor vessel 320 when coupled to the set of reactor racks 306a-306f. In one example, the removable biofilm support microstructures can be uniformly distributed along a peripheral perimeter of the reactor vessel 320 when each of the removable biofilm support microstructures are separated from each adjoining biofilm support microstructure by substantially the same distance. In another aspect, the removable biofilm support microstructures can be uniformly distributed along a peripheral perimeter of the reactor vessel 320 when each removable biofilm support microstructure is separated from a center of the reactor vessel 320 by substantially the same distance. In one example, the variance in distance between each adjoining biofilm support microstructure can be less than about 5%. In one example, the variance in distance between each adjoining biofilm support microstructure can be less than about 1%. In one example, the variance in distance between each biofilm support microstructure and the center of the reactor vessel can be less than about 5%. In one example, the variance in distance between each biofilm support microstructure and the center of the reactor vessel can be less than about 1%.

In one aspect, the reactor vessel can be shaped and sized to fit a base holder so that each complementary magnetic feature or magnetic feature of each reactor rack is magnetically coupled with each complementary magnetic feature or magnetic feature of the base holder. The reactor vessel can comprise any suitable material including glass, polymer, ceramic, or metal.

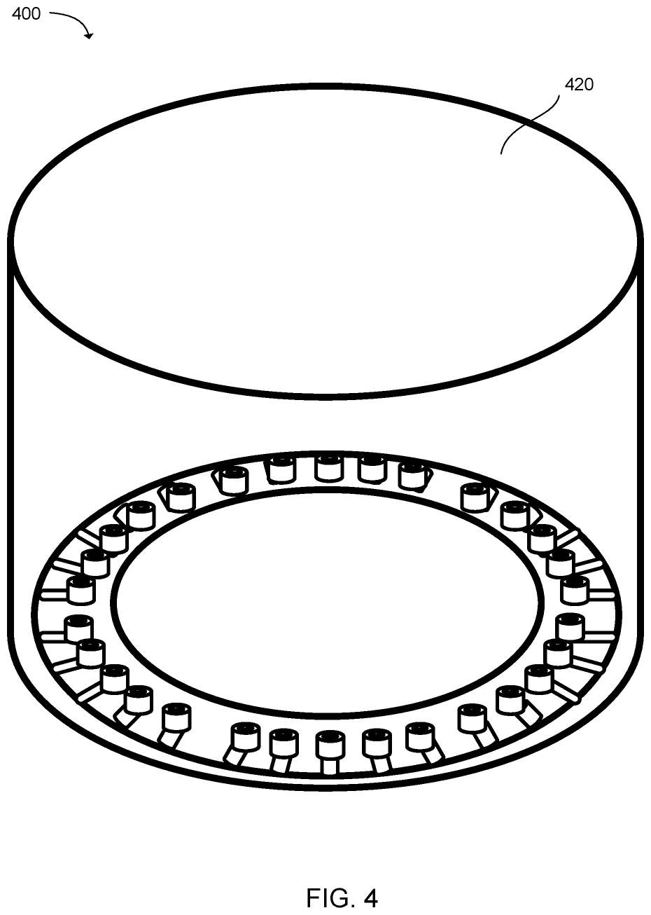

FIG. 4 is a perspective view of biofilm reactor substrate support system 400 with a reactor vessel 420 and the set of reactor racks, 30 pins on each reactor rack, and mounted removable biofilm support microstructures on each of the pins. Each of the reactor racks can include a holding tab. The number of reactor racks can vary between 1 and 60 reactor racks based on the arc length of each reactor rack. In one example, the set of reactor racks can be 2 reactor racks in which each reactor rack has an arc length of about 180°. In one example, the set of reactor racks can be 3 reactor racks in which each reactor rack has an arc length of about 120°. In one example, the set of reactor racks can be 6 reactor racks in which each reactor rack has an arc length of about 60°. In another example, the set of reactor racks can be 12 reactor racks in which each reactor rack has an arc length of about 30°. In another example, the set of reactor racks can be 24 reactor racks in which each reactor rack has an arc length of about 15°. In another example, the set of reactor racks can be 36 reactor racks in which each reactor rack has an arc length of about 10°.

The number of reactor racks can be independently selected apart from the number of reactor racks that forms a complete circle or shape. For example, when each reactor rack has an arc length of about 60° and forms a complete circle when 6 reactor racks are connected together, the number of reactor racks in an experiment can be limited to any number of reactor racks between 1 and 6. As the reactor can be made with varying circumferences, additional racks and/or sizes thereof can be incorporated.

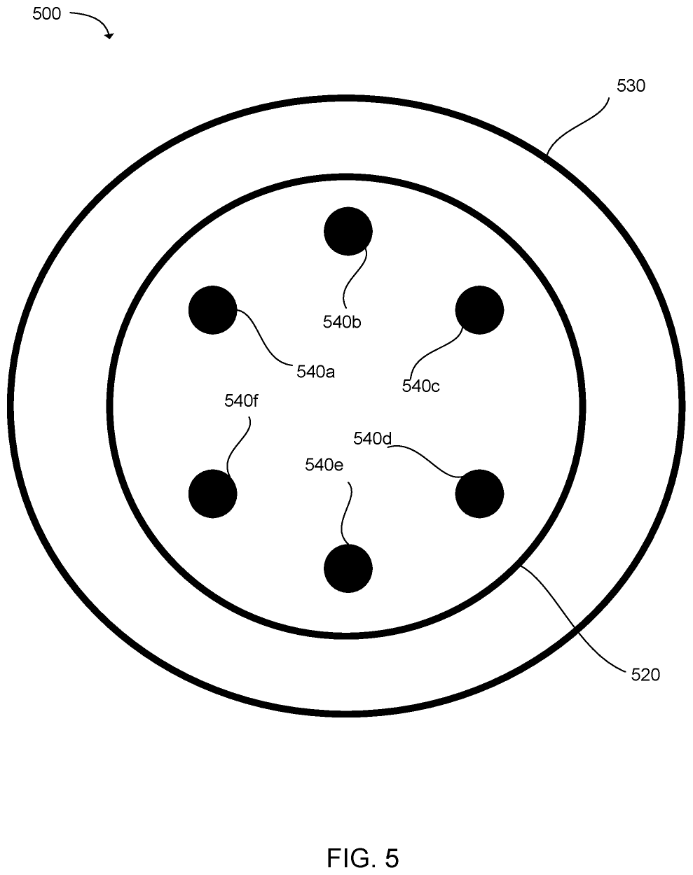

In another example, FIG. 5 illustrates biofilm reactor substrate support system 500 with a base holder 530 for holding the reactor vessel 520 in which the black dots mark the location of the magnetic alignment features 540a-540f In one example, a biofilm reactor substrate support system 500 can comprise a base holder 530 shaped to retain a reactor vessel 520. In one aspect, the base holder 530 can include a magnetic alignment feature (e.g., 540a-540f). In one aspect, the magnetic alignment feature 540a-540f can include embedded neodymium magnets.

In one example, the number of magnetic alignment features of the base holder 530 can vary between 1 and about 36 based on the arc length of reach reactor rack. For example, when the arc length of each reactor rack is about 60°, the number of magnetic alignment features can be 6 so that each magnetic alignment feature can be associated with each reactor rack. In another example, the base holder can have a shape with a perimeter rather than a circumference. In this example, the arc length can be replaced by a partial perimeter that is determined from the ratio of a perimeter of a reactor rack compared to the perimeter when all of the reactor racks are joined together to form a complete set of reactor racks along the an entire periphery.

In one example, the magnetic alignment features can be positioned around a peripheral perimeter or any sub-perimeter of a base holder 530. In one example, when the peripheral perimeter of base holder 530 has a radius of about 50 mm, each of the magnetic alignment features can be positioned about 40 mm from the center of the base holder 530. In another example, each of the magnetic alignment features can be positioned about 30 mm from the center of the base holder 530. In another example, each of the magnetic alignment features can be positioned any distance from the center of the base holder 530 that allows each of the magnetic alignment features to magnetically couple with a complementary magnetic feature of a set of reactor racks.

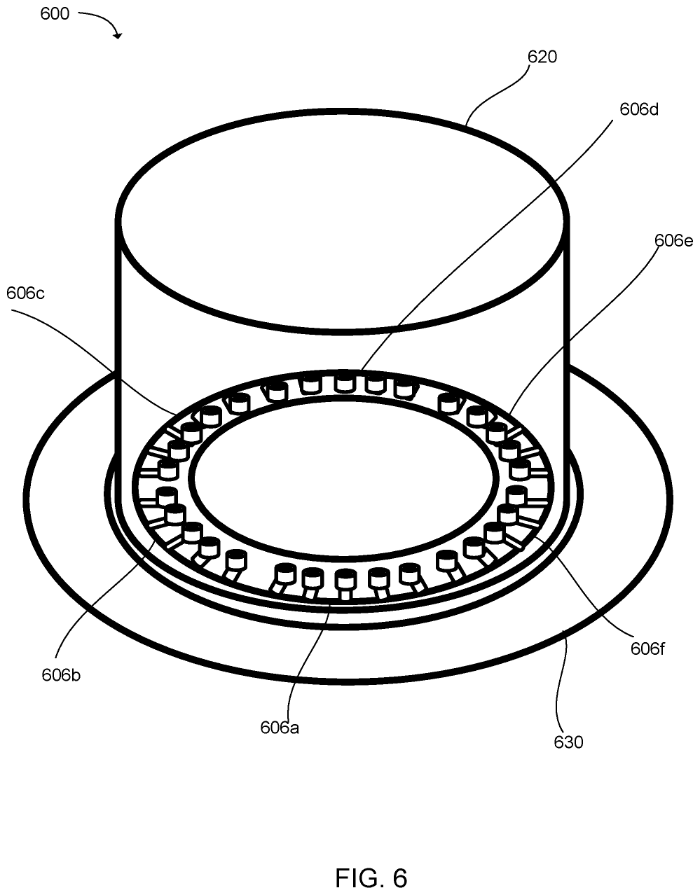

In another aspect, as illustrated in FIG. 6, the biofilm reactor substrate support system 600 can comprise a set of reactor racks (e.g., 606a, 606b, 606c, 606d, 606e, and 606f). The set of reactor racks can be positioned around a periphery of the reactor vessel 620. In one example, the reactor racks can be positioned around a peripheral perimeter or any sub-perimeter of a base holder reactor vessel 620. In one example, when the peripheral perimeter of the reactor vessel 620 has a radius of about 50 mm, each of the reactor racks 606a-606f can be positioned about 40 mm from the center of the reactor vessel 620. In another example, each of the reactor racks 606a-606f can be positioned about 30 mm from the center of the reactor vessel 620. In another example, each of the reactor racks 606a-606f can be positioned any distance from the center of the reactor vessel 620 that allows each of the magnetic alignment features to magnetically couple with a complementary magnetic feature of the base holder.

In another example, the reactor vessel can have a shape with a perimeter rather than a circumference. In this example, the arc length can be replaced by a partial perimeter that is determined from the ratio of a perimeter of a reactor rack compared to the perimeter when all of the reactor racks are joined together to form a complete set of reactor racks along the an entire periphery of the reactor vessel 620.

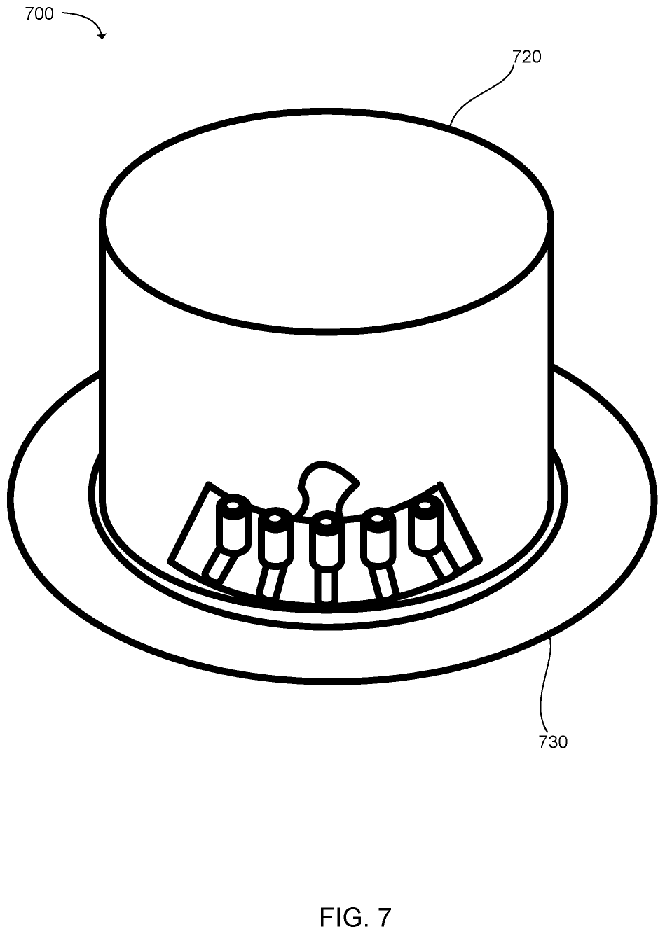

In another embodiment, as illustrated in FIG. 7, the biofilm reactor substrate support system 700 can comprise a reactor rack coupled to at least one removable biofilm support microstructure. Each individual reactor rack can be coupled to the base holder 730 with the reactor vessel 720 positioned intermediate. The reactor rack can be secured in place using a complementary magnetic feature or magnetic feature of the reactor rack that can magnetically couple with a magnetic feature of complementary magnetic feature of the base holder 730. Securing the reactor rack without the presence of any adjoining reactor racks can produce experimental results with consistent environmental conditions such as fluid shear.

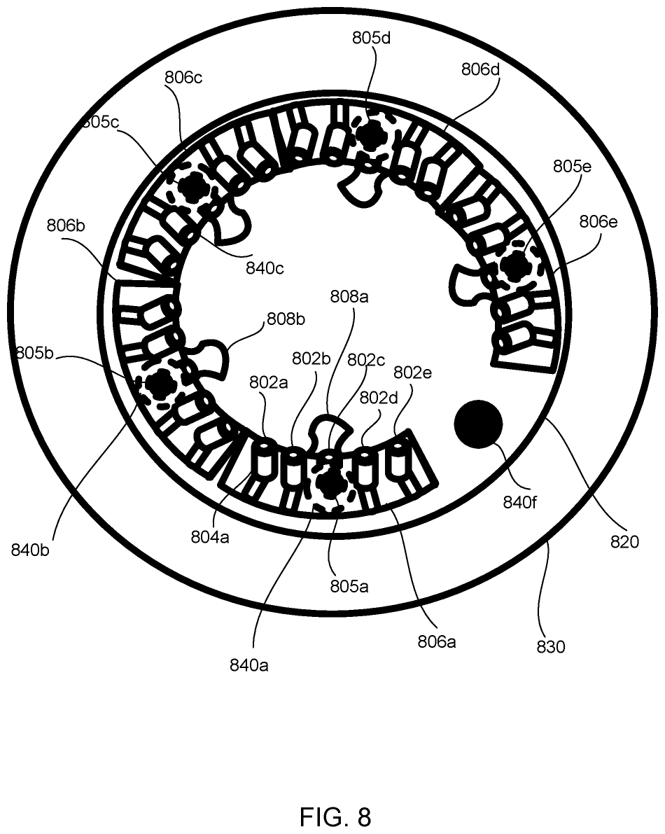

In another example, as illustrated in FIG. 8, each individual reactor rack (e.g., 806a, 806b, 806c, 806d, 806e) can be connectable with at least one adjacent reactor rack (e.g., 806a can be adjacent to 806b; 806b can be adjacent to 806a and 806c, and so forth) in the set of reactor racks 806a-806e. In one aspect, the set of reactor racks 806a-806e can be configured to be uniformly distributed inside the reactor vessel 820 when each reactor rack 806a-806e is connected with the adjacent reactor rack (e.g., 806a, 806b, 806c, 806d, or 806e). In one aspect, each reactor rack can be configured to be connected to adjacent reactor racks using one or more of magnetic coupling, slotted coupling, snaps, interference fitting, adhesion, threading, the like, and combinations thereof.

Each reactor rack 806a-806e can have an arcuate shape that matches a bottom perimeter of the reactor vessel 820. In one example, each reactor rack can have a partial shape that forms a complete shape when each reactor rack is connected into a set of reactor racks. The partial shape can include a symmetrical shape including one or more of: a triangle, a square, a rectangle, a regular polygon, a circle, an ellipse, the like, and combinations thereof. Each reactor rack 806a-806e can comprise a material including at least one of glass, polymer, ceramic, and metal. In one aspect, the material can include stainless steel.

The set of reactor racks 806a-e can include a complementary magnetic alignment feature (e.g., 805a, 805b, 805c, 805d, and 805e) which can be magnetically coupled to the magnetic alignment feature 840a, 840b, 840c 840d, 840e, and 840f of the base holder 830 so as to anchor the base holder 830 and the set of reactor racks 806a-e in a fixed position relative to one another. In one example, the set of reactor racks 806a-806e can include between about 2 and about 10 reactor racks. In another example, the set of reactor racks can include between about 2 and 60 reactor racks. The number of reactor racks, the number of complementary magnetic alignment features of each reactor rack, and the number of magnetic alignment features of the base holder can be the same so that each reactor rack can be magnetically coupled to each magnetic alignment feature of the base holder.

In another example, the magnetic alignment feature (e.g., 840a, 840b, 840c 840d, 840e, and 840f) can be a set of permanent magnets and the complementary magnetic alignment feature (e.g., 805a-805e) can be at least one of ferromagnetic material and ferrimagnetic material. The set of reactor racks 806a-806e can be either formed of or incorporate the complementary magnetic alignment feature (e.g., 805a-805e).

In another example, the complementary magnetic alignment feature (e.g., 840a-840f) can be a set of permanent magnets and the magnetic alignment feature (e.g., 805a-805e) can be at least one of ferromagnetic material and ferrimagnetic material. In one aspect, the base holder 830 can be either formed of or incorporate the complementary magnetic alignment feature (e.g., 840a-840f).

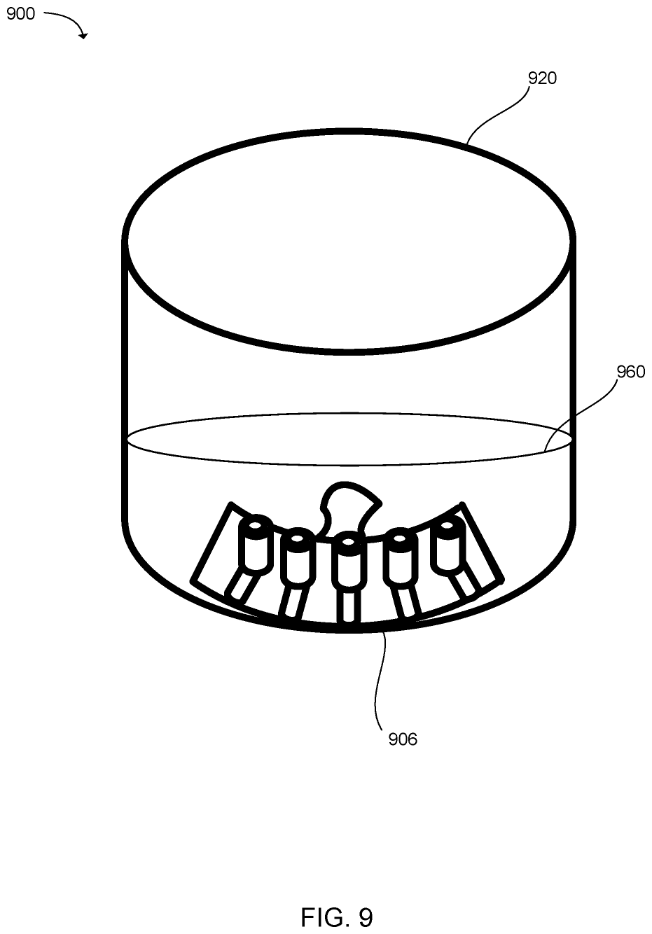

FIG. 9 illustrates a biofilm reactor substrate support system 900 including biofilms on the removable biofilm support microstructures on a reactor rack 906 in a reactor vessel 920 after a time period of growth. The reactor rack 906 can be submerged in a liquid 960 including various media and reagants including but not limited to one or more of: culture media, buffer, plating media, ethanol, the like, and combinations thereof.

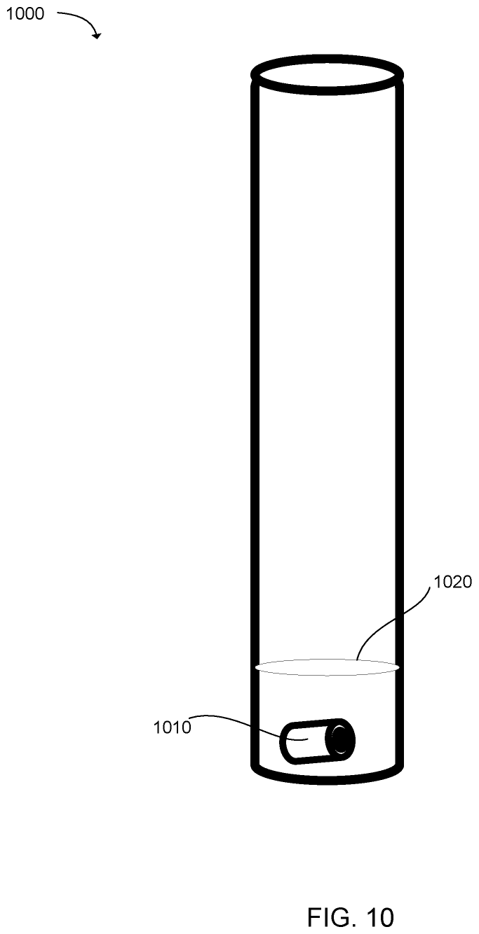

FIG. 10 illustrates a removable biofilm support microstructure 1010 and associated biofilms in a test tube 1000 for biofilm quantification. The removable biofilm support microstructure 1010 can be submerged in a liquid 1020 including various media and reagants including but not limited to one or more of: culture media, buffer, plating media, ethanol, the like, and combinations thereof.

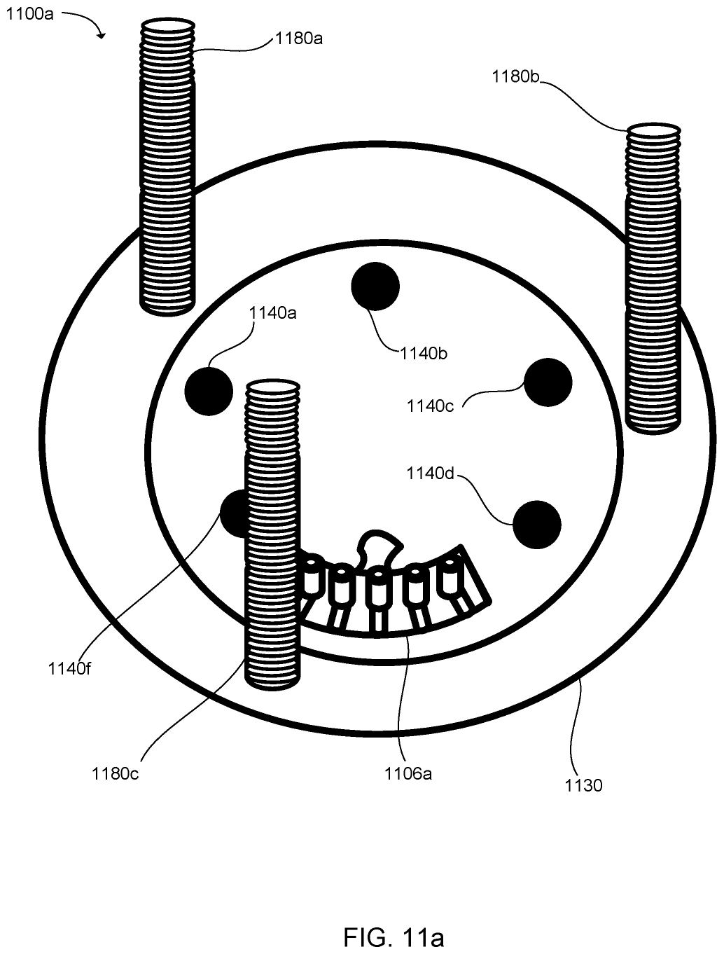

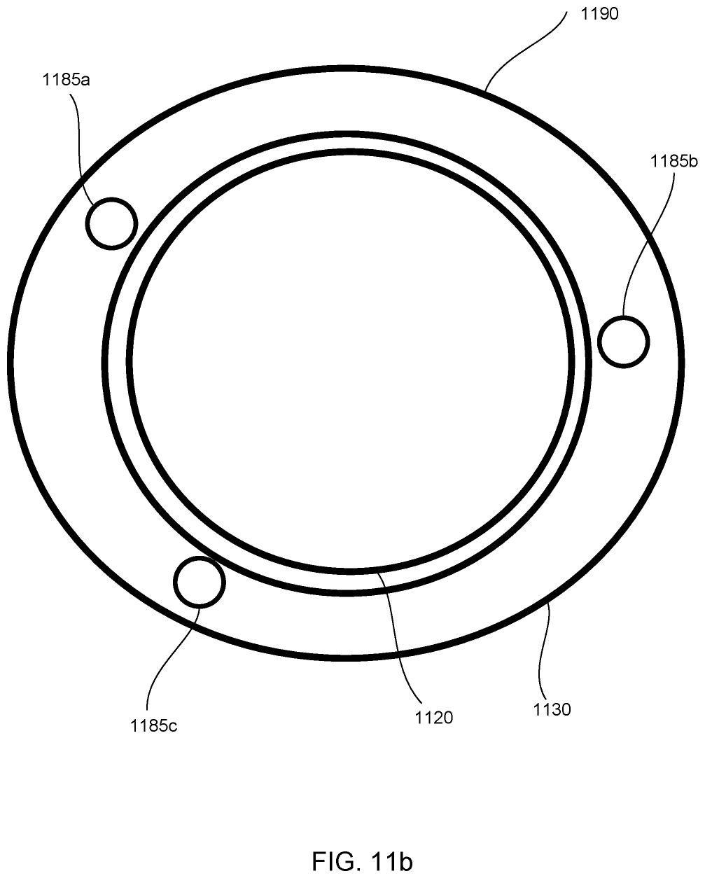

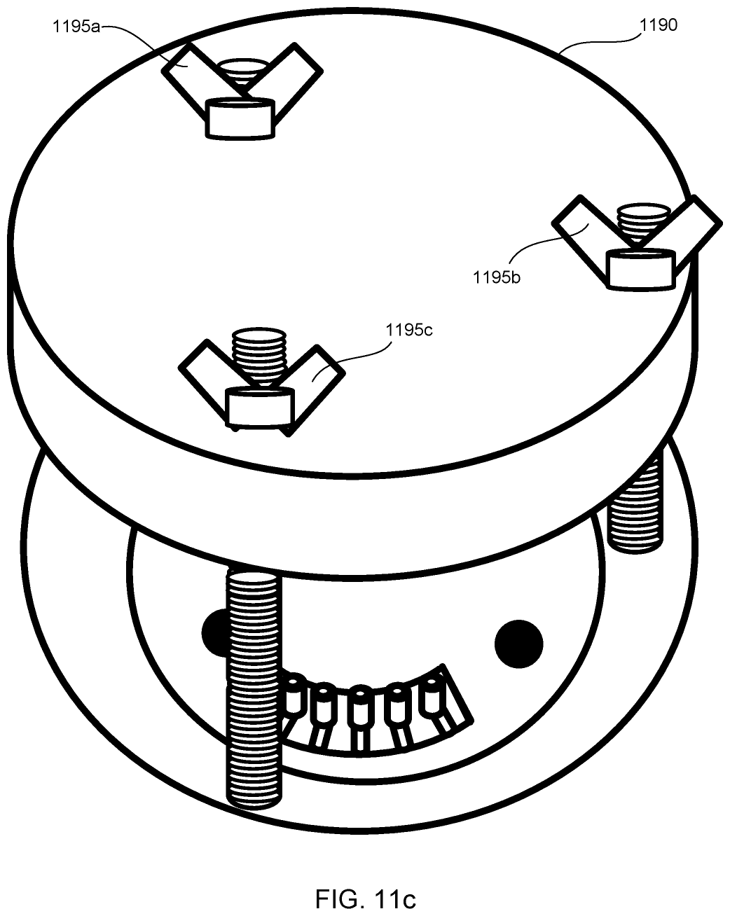

In another embodiment, as illustrated in FIG. 11a, a biofilm reactor substrate support system 1100a can include a base holder 1130, a reactor rack 1106a, magnetic alignment features 1140a-1140f, and threaded shafts 1180a, 1180b, and 1180c. The magnetic alignment features 1140a-1140f can be magnetically coupled to complementary magnetic alignment features of the reactor racks. The threaded shafts 1180a, 1180b, and 1180c can interface with a set of threaded retaining rings.

In another aspect, as illustrated in FIG. 11b, a top holder 1190 can include a slot 1120b that can interface with a top of a reactor vessel 1120. The top holder 1190 can include a set of threaded retaining rings 1185a, 1185b, and 1185c that can interface with the threaded shafts 1180a, 1180b, and 1180c. In another aspect, as illustrated in FIG. 11c, the top holder 1190 can be secured to the threaded shafts using wing nuts 1195a, 1195b, and 1195c.

FIGS. 11a-11c are presented by way of example and the base holder 1130 and the top holder 1190 can be coupled using various methods including magnetic coupling, slotted coupling, snaps, interference fitting, adhesion, threading, suction cup, and the like, and combinations thereof.

The racks can alternatively be formed in a variety of shapes. For example, racks can be formed as concentric rings, a circular plate, or as a polygon. A segmented circular plate can orient the reactor substrates or beads circumferentially equidistant from a center of the circle. Similarly, a segmented equiangular polygon can have reactor substrates oriented equidistant from the center, or varied in distance from the center in each rack, but having the same radial spacing among each rack in the segmented supports.

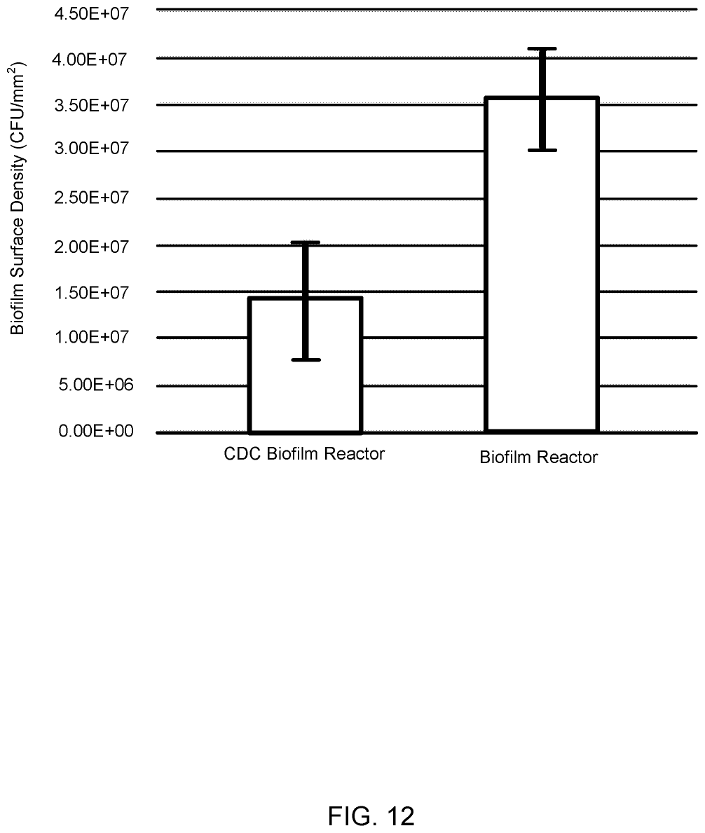

FIG. 12 illustrates a flow diagram of a method according to the present technology. For simplicity of explanation, the method is depicted and described as a series of acts. However, acts in accordance with this disclosure can occur in various orders and/or concurrently, and with other acts not presented and described herein. Furthermore, not all illustrated acts may be required to implement the methods in accordance with the disclosed subject matter.

In one example, a method 1300 for screening anti-biofilm compounds is provided. The method can include anchoring a set of reactor racks in a fixed position to a base holder using a magnetic alignment feature and a complementary magnetic alignment feature, as shown in block 1310. The method can further include growing biofilm on a removable biofilm support structure coupled to a reactor rack of the set of reactor racks, as shown in block 1320. The method can further include separating the removable biofilm support structure and the reactor rack from the set of reactor racks, as shown in block 1330. The method can further include treating the biofilm on the removable support structure with an anti-biofilm compound, as shown in block 1340.

Although anti-biofilm compounds can be readily screened with this reactor, other applications can include, but are not limited to, physiology, physiochemistry, growth parameters, morphology, etc.

### Example

In one example, the biofilm reactor of this application can use a reactor vessel for use with an orbital shaker and incubator. It can have the same diameter as a standard culture plate (e.g., 100 mm) and can therefore fit into a standard anaerobic jar used by microbiologists. The reactor vessel can contain 6 racks in which each rack can have the footprint of an arc. The 6 racks can form a complete 360-degree ring around the outer edge of the reactor vessel. The arc racks can be made from ferromagnetic stainless steel which can be autoclaved. Each rack can have 5 vertical pins which can receive beads upon which the biofilms are grown. The number of racks, vertical pins, and beads can be varied.

In another example, the 30 vertical pins and the 30 mounted beads from the 6 racks can be uniformly oriented around the outer edge of the reactor vessel such that each of the mounted beads can experience identical fluid shear (i.e. force opposing flow) when placed on the orbital shaker. The reactor can hold about 50 ml of broth, although the size of the reactor can be adjusted. In some cases, a robust Staphylococcus aureus (ATCC 6538) can be grown in about 48 hours with three broth exchanges of 50 ml for a total of 150 ml. These biofilms can have greater surface area density than equivalent 48-hour biofilms grown in a CDC biofilm reactor with the same isolate using a total of 13.5 liters of broth.

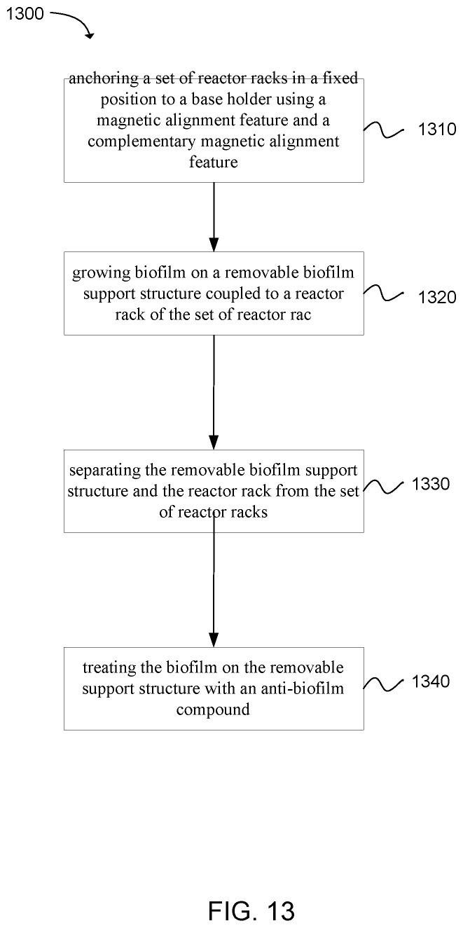

As depicted in FIG. 13, the biofilm reactor produced biofilms with a surface area density of 3.56×10⁷±5.15×10⁶ CFU/mm² compared with the CDC biofilm reactor's surface area density of 1.42×10⁷±6.13×10⁶ CFU/mm². The biofilm reactor also included a base holder for holding the 100-mm reactor vessel. The base holder was strategically embedded with 6 neodymium magnets coinciding with the center of each rack. The 6 neodymium magnets enabled the biofilm contents of the reactor, once grown up to maturity, to be transferred on a rack including 5 removable biofilm support microstructures to separate PYREX reactor vessels and base holders for varied treatments. The magnetic base kept the racks firmly anchored to the outer margin of the treatment dish, without using neighboring racks to lock the system in place. Consequently, at the time of treatment, only one variable (i.e. antibiotic treatment) was altered which allowed the biofilms to reside in the same shear profiles and environmental conditions. Each rack was also equipped with a holding tab to facilitate moving the racks for treatment and transferring the biofilm beads for quantification at the end of the experiment.

While the flowcharts presented for this technology may imply a specific order of execution, the order of execution may differ from what is illustrated. For example, the order of two more blocks may be rearranged relative to the order shown. Further, two or more blocks shown in succession may be executed in parallel or with partial parallelization. In some configurations, one or more blocks shown in the flow chart may be omitted or skipped.

Reference throughout this specification to “an example” means that a particular feature, structure, or characteristic described in connection with the example is included in at least one embodiment of the present invention. Thus, appearances of the phrases “in an example” in various places throughout this specification are not necessarily all referring to the same embodiment.

Reference was made to the examples illustrated in the drawings, and specific language was used herein to describe the same. It will nevertheless be understood that no limitation of the scope of the technology is thereby intended. Alterations and further modifications of the features illustrated herein, and additional applications of the examples as illustrated herein, which would occur to one skilled in the relevant art and having possession of this disclosure, are to be considered within the scope of the description.

Furthermore, the described features, structures, or characteristics may be combined in any suitable manner in one or more examples. In the preceding description, numerous specific details were provided, such as examples of various configurations to provide a thorough understanding of examples of the described technology. One skilled in the relevant art will recognize, however, that the technology may be practiced without one or more of the specific details, or with other methods, components, devices, etc. In other instances, well-known structures or operations are not shown or described in detail to avoid obscuring aspects of the technology.

Although the subject matter has been described in language specific to structural features and/or operations, it is to be understood that the subject matter defined in the appended claims is not necessarily limited to the specific features and operations described above. Rather, the specific features and acts described above are disclosed as example forms of implementing the claims. Numerous modifications and alternative arrangements may be devised without departing from the spirit and scope of the described technology.

The foregoing detailed description describes the invention with reference to specific exemplary embodiments. However, it will be appreciated that various modifications and changes can be made without departing from the scope of the present invention as set forth in the appended claims. The detailed description and accompanying drawings are to be regarded as merely illustrative, rather than as restrictive, and all such modifications or changes, if any, are intended to fall within the scope of the present invention as described and set forth herein.

What is claimed is:

1. A biofilm reactor substrate support system comprising:
a base holder shaped to retain a reactor vessel and comprising a magnetic alignment feature; and
a set of reactor racks including a complementary magnetic alignment feature which is magnetically coupleable to the magnetic alignment feature so as to anchor the base holder and the set of reactor racks in a fixed position relative to one another,
wherein each reactor rack is coupleable to at least one removable biofilm support microstructure.

2. The biofilm reactor substrate support system of claim 1, wherein the set of reactor racks comprises 2 to 30 reactor racks.

3. The biofilm reactor substrate support system of claim 1, wherein the set of reactor racks is coupleable to the base holder with the reactor vessel positioned intermediate.

4. The biofilm reactor substrate support system of claim 1, wherein each reactor rack is connectable with an adjacent reactor rack in the set of reactor racks.

5. The biofilm reactor substrate support system of claim 4, wherein the set of reactor racks are configured to be uniformly distributed inside the reactor vessel when each reactor rack is connected with the adjacent reactor rack.

6. The biofilm reactor substrate support system of claim 1, wherein each reactor rack has an arcuate shape that matches a bottom perimeter of the reactor vessel.

7. The biofilm reactor substrate support system of claim 1, wherein each reactor rack comprises stainless steel.

8. The biofilm reactor substrate support system of claim 1, wherein the magnetic alignment feature is a set of permanent magnets and the complementary magnetic alignment feature is at least one of ferromagnetic material and ferrimagnetic material, where the set of reactor racks is either formed of or incorporates the complementary magnetic alignment feature.

9. The biofilm reactor substrate support system of claim 1, wherein the complementary magnetic alignment feature is a set of permanent magnets and the magnetic alignment feature is at least one of ferromagnetic material and ferrimagnetic material, where the base holder is either formed of or incorporates the complementary magnetic alignment feature.

10. The biofilm reactor substrate support system of claim 1, further comprising the at least one removable biofilm support microstructure.

11. The biofilm reactor substrate support system of claim 10, wherein the at least one removable biofilm support microstructure has a width on the order of 1-100 mm.

12. The biofilm reactor substrate support system of claim 10, wherein the at least one removable biofilm support microstructure is shaped as at least one of: a bead, a cylinder, a sphere, a torus, an ellipsoid, a diamond, or a helix.

13. The biofilm reactor substrate support system of claim 10, wherein the at least one removable biofilm support microstructure is radially symmetric.

14. The biofilm reactor substrate support system of claim 10, wherein the at least one removable biofilm-support microstructure comprises one or more of glass, polymer, ceramic, or metal.

15. The biofilm reactor substrate support system of claim 10, wherein the at least one removable biofilm support microstructure comprises between about 1 and about 100 removable biofilm support microstructures.

16. The biofilm reactor substrate support system of claim 15, wherein the at least one removable biofilm support microstructures are configured to be uniformly distributed along a peripheral perimeter of the reactor vessel when coupled to the set of reactor racks.

17. The biofilm reactor substrate support system of claim 10, further comprising a mount configured to receive the removable biofilm support microstructure.

18. The biofilm reactor substrate support system of claim 17, wherein the mount is a vertical pin and the removable biofilm support microstructures include a receiver hole sized to be mounted on the vertical pin.

19. The biofilm reactor substrate support system of claim 10, wherein each reactor rack is coupleable to at least one removable biofilm support microstructure using at least one or more of magnetic coupling, slotted coupling, snaps, interference fitting, adhesion, and threading.

20. The biofilm reactor substrate support system of claim 1, wherein the at least one removable biofilm support microstructure provides a biofilm surface area of 1 to 1000 mm² and a reactor volume of 10 to 2000 ml.

21. The biofilm reactor substrate support system of claim 1, further comprising the reactor vessel configured to contain the set of reactor racks.

22. The biofilm reactor substrate support system of claim 1, wherein the set of reactor racks each include a holding tab configured to facilitate movement of the ferromagnetic reactor rack.

23. A method for screening anti-biofilm compounds comprising:
anchoring a set of reactor racks in a fixed position to a base holder using a magnetic alignment feature and a complementary magnetic alignment feature;
growing biofilm on a removable biofilm support structure coupled to a reactor rack of the set of reactor racks;
separating the removable biofilm support structure and the reactor rack from the set of reactor racks; and
treating the biofilm on the removable support structure with an anti-biofilm compound.
